SEQUENCE LISTING (3 sequences)

SEQ ID NO: 1 - DNA, 173 bp, Artificial Sequence
  Exon 1 of an antisense having type I collagenase (MMP-1) inhibiting activity
atattggagtagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccag

SEQ ID NO: 2 - DNA, 483 bp, Artificial Sequence
  Inton 1 of an antisense having type I collagenase (MMP-1) inhibiting activity
gtaaatgctgcattgcgtgtgacaataatgtaaatttttagtttgtatttttctgcagtaatgtaatagagttttttaaggataggtttcttataaagagattttttttttttgctagaaacagcaccctccacccaaaatgtatctagccatgcatacgctctctttttccagttggaggtgagagtgaactaaaggaacaacatccaaacttgtctccacaaattgtagacttgtaacagtatgcaaatcttgctctacaaaaattgctctactatgaagttcttacttcacaataactaatagtagggacatttctactctgaaatttcatttctcaccaaaaataaggataaaagcaactgcgaaatctaaacacaaggtttaaaagcagttctctgttttggctaaaacttaatgccatcaatttttagaataatgagaaagatttcccatgcagtgtttcgataattttcttttgtc

SEQ ID NO: 3 - DNA, 105 bp, Artificial Sequence
  Exon 2 of an antisense having type I collagenase (MMP-1) inhibiting activity
aaatacctggaaaaatactacaacctgaagaatgatgggaggcaagttgaaaagcggagaaatagtggcccagtggttgaaaaattgaagcaaatgcaggaattc

(19) United States
(12) Patent Application Publication
(10) Pub. No.: US 2003/0108530 A1
(43) Pub. Date: Jun. 12, 2003

(54) Antisense inhibiting melanoma invasion and functional analogs thereof

(76) Inventors: Pnina Brodt, Montreal (CA); Margaret Durko, Cote St-Luc (CA)

Correspondence Address: KLAUBER & JACKSON, 411 HACKENSACK AVENUE, HACKENSACK, NJ, 07601

(21) Appl. No.: 10/247,800

(22) Filed: Feb. 11, 2003

Related U.S. Application Data
(63) Continuation of application No. PCT/CA01/00366, filed on Mar. 19, 2001.
(60) Provisional application No. 60/190,088, filed on Mar. 20, 2000.

(51) Int. Cl. A61K 48/00; C07H 21/04; C12N 15/85

(52) U.S. Cl. 424/93.21; 514/44; 435/455; 435/320.1; 536/23.2

(57) ABSTRACT

The present invention relates to an antisense to inhibit melanoma invasion, to an expression vector and to a method for substantially inhibiting tumor cell invasion of an extracellular matrix (ECM) and other invasive processes such as angiogenesis, and particularly plasmin-mediated proteolysis thereof in a patient, by suppression of type I collagenase (MMP-1) expression in the patient.

### BACKGROUND OF THE INVENTION

(a) Field of the Invention

The present invention relates to an antisense to inhibit melanoma invasion, to an expression vector comprising same and to a method for substantially inhibiting tumor cell invasion of an extracellular matrix (ECM) and particularly plasmin-mediated proteolysis thereof in a patient, by suppressing expression or function of type I collagenase (MMP-1) in the patient.

(b) Description of the Prior Art

The extracellular matrix (ECM) is a complex structure consisting mainly of basement membranes and interstitial stroma and composed of collagen, glycoproteins and proteoglycans, forming a dense meshwork normally impenetrable to migrating cells. ECM turnover is essential for normal physiological processes such as organogenesis and wound healing. In pathological processes requiring degradation of ECM such as cancer invasion and metastasis, the tight regulation of ECM turnover is disrupted, leading to increased ECM proteolysis. Several different classes of proteinases are known to participate in ECM degradation. These include matrix metalloproteinases (MMPs) such as type I (MMP-1) and type IV (MMP-2, MMP-9) collagenases and stromelysin-1 (MMP-3), and serine proteinases such as the urokinase-type plasminogen activator (uPA) and plasmin. These enzymes have all been implicated in cancer invasion and metastasis (Mignatti, P., and Rifkin, D. B. (1993) Physiol. Rev. 73, 161-195).

The uPA enzyme converts the zymogen plasminogen to its enzymatically active form plasmin. Plasmin in turn can initiate the conversion of the uPA zymogen (pro-uPA) to its active form uPA, resulting in an autocatalytic loop. Activation of plasminogen to plasmin occurs at the cell surface where uPA binds through a specific cell surface receptor (urokinase-type plasminogen activator receptor, or uPAR) and plasminogen binds through as yet unidentified binding sites. Receptor-bound uPA can be inactivated by the plasminogen activator inhibitors PAI-1 and PAI-2. Binding of the ECM-associated inhibitor PAI-1 to the receptor-linked uPA in turn triggers the internalization of the whole complex and the reexpression of the receptor at new sites. This provides a mechanism for coordinated regulation of uPAR turnover, cell surface plasminogen activation and cellular migration (Mignatti, P., and Rifkin, D. B. (1993) Physiol. Rev. 73, 161-195).

Plasmin contributes to ECM degradation both directly and indirectly. It is a broad-spectrum proteinase which can degrade most components of the ECM including proteoglycans and glycoproteins (i.e. laminin, fibronectin) present in the extracellular matrix (Mignatti, P., and Rifkin, D. B. (1993) Physiol. Rev. 73, 161-195), and possibly some types of collagen. Plasmin can activate MMP zymogens through amino-terminal processing (He, C., Wilhelm, S. M., Pentland, A. P., Marmer, B. L., Grant, G. A., Eisen, A. Z., and Goldberg, G. I. (1989) Proc. Natl. Acad. Sci. 86, 2632-2636) either in its cell-bound form or in the extracellular milieu. Plasmin has been identified as an important MMP-1 activator and a regulator of its synthesis (Lee, E., Vaughan, D. E., Parikh, S. H., Grodzinski, A. J., Libby, P., Lark, M. W., and Lee, R. T. (1996) Circ. Res. 78, 44-49).

The uPA receptor (uPAR) is a key component of the plasminogen activation pathway (Roldan, A. L., Cubellis, M. V., Masucci, M. T., Behrendt, N., Lund, L. R., Dano, K., Appella, E., and Blasi, F. (1990) EMBO J. 9, 467-474). Increased expression of uPAR has been noted on the surface of migrating cells and on highly invasive or metastatic tumor cells (de Vries, T. J., Quax, P. H. A., Denijn, M., Verrijp, K. N., Verheijen, J. H., Verspaget, H. W., Weidle, U. H., Ruiter, D. J., and van Muijen, G. N. P. (1994) Am. J. Pathol. 144, 70-81). Expression of uPAR can be regulated by various exogenous stimuli including cytokines, growth factors and phorbol esters (Lengyel, E., Wang, H., Stepp, E., Juarez, J., Wang, Y., Doe, W., Pfarr, C. M., and Boyd, D. (1996) J. Biol. Chem. 271, 23176-23184). An AP-1 consensus sequence has been identified in the uPAR promoter 184 base pairs upstream of the transcriptional start site (Lengyel, E., Wang, H., Stepp, E., Juarez, J., Wang, Y., Doe, W., Pfarr, C. M., and Boyd, D. (1996) J. Biol. Chem. 271, 23176-23184). This transcriptional element was also identified in the promoter regions of several metalloproteinases (Durko, M., and Brodt, P. (1996) In Cell Adhesion and Invasion in Cancer Metastasis. (Brodt, P. Ed.), pp. 113-150, R. G. Landes Company, Medical Intelligence Unit, Georgetown, Tex.), among them MMP-1, suggesting that transcription of uPAR and some collagenases could be subject to regulation by similar mechanisms. However, the link between uPAR and collagenase transcription and regulation remains, to this date, unknown.

Melanoma cell invasion and metastasis require degradation of interstitial stroma and basement membranes of the ECM. These processes have been shown to depend on proteolytic cascades involving the MMPs and the plasminogen/plasmin system (Lee, E., Vaughan, D. E., Parikh, S. H., Grodzinski, A. J., Libby, P., Lark, M. W., and Lee, R. T. (1996) Circ. Res. 78, 44-49; de Vries, T. J., Quax, P. H. A., Denijn, M., Verrijp, K. N., Verheijen, J. H., Verspaget, H. W., Weidle, U. H., Ruiter, D. J., and van Muijen, G. N. P. (1994) Am. J. Pathol. 144, 70-81). Metastasis is a complex multistep process during which tumor cells invade through different ECMs such as basement membrane and connective tissue, and give rise to new foci at sites distant from the primary tumor. The tumor cell anchors to the ECM via cell surface receptors. The anchored tumor cell next secretes the hydrolytic enzymes which degrade the ECM and causes lysis thereof. The tumor cell then migrates through the ECM.

In melanoma cells, the upregulation of MMP-1 during malignant progression can provide the tumor cell with a proteolytic mechanism for dissolution of dermal collagen. In addition, it can also enhance degradation of subendothelial basement membranes and facilitate invasion into blood vessel and dissemination by regulating the activity of other metalloproteinase. Human melanoma subclones were previously obtained, in which MMP-1 expression was inhibited (up to 96%) by antisense RNA, and the invasive capability of these clones as measured in a Matrigel-invasion system was lost (Durko, M., Navab, R., Shibata, H., and Brodt, P. (1997) Biochim. Biophys. Acta, 1356, 271-280). Durko et al. describe the in vitro suppression of MMP-1 expression in human melanoma MIM cells originally derived from a metastasis. In these cells, invasion through type IV collagen-containing matrix was inhibited although type IV collagenase (MMP-2) expression levels were unaltered.

There is compelling evidence to indicate that cell migration and invasion depend on the coordinated enzymatic activities of metallo- and serine proteinases. Colocalization of metalloproteinases (Moll, U. M., Lane, B., Zucker, S., Suzuki, K., and Nagase, H. (1990) Cancer Res. 50, 6995-7002), serine proteinases and the uPA receptor (de Vries, T. J., Quax, P. H. A., Denijn, M., Verrijp, K. N., Verheijen, J. H., Verspaget, H. W., Weidle, U. H., Ruiter, D. J., and van Muijen, G. N. P. (1994) Am. J. Pathol. 144, 70-81) in microvilli-like projections at the tumor-stroma interface of invading cells has been documented in vivo. In vitro, tumor cell invasion into reconstituted basement membrane (Matrigel) or amnion were shown to depend on a proteolytic cascade involving metallo- and serine proteinases (Mignatti, P., Robbins, E., and Rifkin, D. B. (1986) Cell 47, 487-498). Of particular interest to this report are findings of coordinated activities of MMP-1 and plasmin in cultured skin fibroblasts and keratinocytes (He, C., Wilhelm, S. M., Pentland, A. P., Marmer, B. L., Grant, G. A., Eisen, A. Z., and Goldberg, G. I. (1989) Proc. Natl. Acad. Sci. 86, 2632-2636) and in invading carcinoma cells. In melanoma cells, plasmin production has been associated with progression to an invasive/malignant phenotype and both MMP-1 and plasmin were implicated in invasion (Mignatti, P., Robbins, E., and Rifkin, D. B. (1986) Cell 47, 487-498).

As most cancer patients fail to respond to treatment due to the development of metastasis, it is desirable to inhibit invasion of the normal surrounding tissue by the tumor cells. However, known invasion inhibitors tested to date have been of limited benefit clinically.

It would therefore be desirable to be provided with an improved invasion inhibitor which would substantially inhibit tumor cell degradation of the surrounding normal tissue and invasion through the extracellular matrix.

### SUMMARY OF THE INVENTION

One aim is to provide an improved invasion inhibitor which substantially inhibits tumor cell mediated degradation of the surrounding normal tissue and invasion through the extracellular matrix.

In accordance with a broad aspect of the present invention, there is provided an antisense having type I collagenase (MMP-1) synthesis-inhibiting activity or a functional analog thereof, which comprises a first nucleotide sequence adapted to hybridize with a cellular RNA transcribed from a second nucleotide sequence encoding a peptide having MMP-1 activity, to substantially reduce MMP-1 synthesis or MMP-1 function and/or inhibit plasmin-mediated proteolysis and/or invasion of an extracellular matrix by a tumor cell in a patient.

The antisense may have from about 18 to about 770 nucleotides and is complementary to a target region of the RNA encoding the peptide having MMP-1 activity. Preferably, the antisense has the nucleotide sequence set forth in SEQ ID NO:1. The exact nucleotide sequence and chemical structure of an antisense according to the present invention can be varied, so long as the antisense retains its ability to substantially inherit MMP-1 expression.

In accordance with another broad aspect of the present invention, there is provided an expression vector comprising such an antisense sequence operably linked to a promoter region.

In accordance with yet another broad aspect of the present invention, there is provided a method for substantially inhibiting plasmin-mediated proteolysis and/or invasion of an extracellular matrix by a tumor cell in a patient. The method comprises substantially inhibiting the function or the expression of type I collagenase (MMP-1) by the tumor cell, thereby substantially reducing plasminogen activation and inhibiting invasion of the extracellular matrix by the tumor cell in the patient.

The methods may be effected by administering to the patient such an antisense in the form of an oligodeoxynucleotide sequence or comprised in a viral vector. The expression of MMP-1 by the tumor cell is substantially inhibited, which reduces levels of membrane-bound plasmin.

By suppressing expression of type I collagenase (MMP-1) in the patient, degradation of types I, II, III and IV collagen, glycoproteins and proteoglycans present in the extracellular matrix can be substantially inhibited.

To inhibit expression of the MMP-1 gene, an antisense nucleotide sequence such as a single-stranded DNA molecule complementary to the mRNA transcribed from the MMP-1 gene is inserted in the target cell. The antisense molecule then hybridizes or base-pairs with the cellular mRNA, thereby preventing translation of the mRNA into a peptide having MMP-1 activity.

The antisense molecule may be microinjected into the target cell or expression vectors such as viral vectors can be used to produce the antisense RNA in transfected cells. The cells can be transduced with a vector carrying the sequence in an antisense orientation downstream of a promoter. The RNA molecule transcribed from the vector is complementary in sequence to the mRNA transcribed from the gene in the target cell, and hybridizes therewith to form a double-stranded RNA, which cannot be translated into a peptide, thereby suppressing expression of MMP-1 gene. Synthetic single-stranded nucleotide sequences can also be inserted in the target cell.

A “tumor cell” is intended to mean a cancerous cell in which MMP-1 is functionally relevant to the process of invasion. Preferably, the tumor cell is a melanoma cell but breast carcinoma cells and osteosarcoma cells can also be targets (Benbow U., Schoenermark M P, Orndorff K A, Givan A L and Brinckerhoff C E (1999), Clin. Exp. Metastasis 17:231-238; Duivenvoorden W C, Hirte H W and Singh G. (1999) Clin. Exp. Metastasis 17:27-34). In addition, normal cells of the patient such as stromal cells which facilitate tumor invasion by producing MMP-1 (Nakopoulou I., Giannopoulou I., Gakiopoulou H., Liapis H., Tzonou A. Davaris P S (1999) Human Pathology 30:436-442) and endothelial cells (Oda N. Abe M. and Sato Y. (1999) J. Cell. Physiol. 178:121-132) can also be targets.

### BRIEF DESCRIPTION OF THE FIGURES

FIG. 1 shows results of an analysis of caseinolytic activities in melanoma cell-conditioned media. Zymographic analysis was performed with concentrated serum-free conditioned media. The proteins (20 μg per lane) were separated by electrophoresis on 10% polyacrylamide gels co-polymerized with 1 mg/ml casein. Shown in lanes 1-4 are results with wild-type MIM and clone 25-2, 25-11 and 25-12 cells, respectively. To identify the caseinolytic activity, the enzymatic reaction was carried out in presence of EDTA (lane 5) or Amino-n-Caproic acid (lane 6), using clones 25-2 and 25-11, respectively. The estimated M.W. (×10⁻³) are shown on the right.

FIG. 2 shows results of a Western blot analysis of plasmin production by melanoma cells. Concentrated serum-free conditioned media derived from wild-type MIM (lanes 1, 2) or clone 25-11 (lane 3) cells (10 μg protein per lane) were subjected to SDS-PAGE using 10% gels. Purified plasminogen (0.1 μg) was used as a control (lane 4). The resolved proteins were transferred to a nitrocellulose membrane and probed with a rabbit antiserum which recognizes both plasminogen and plasmin (lanes 2, 3 and 4) or with normal rabbit serum as a control (lane 1). An alkaline phosphatase-conjugated goat anti-rabbit IgG was used as a second antibody. The estimated M.W. (×10⁻³) is shown on the right.

FIG. 3 shows results of a flow cytometric analysis of cell-surface bound plasminogen. MIM and 25-12 cells were cultured in serum-free medium for 48 hours. The cells were then harvested and 10⁵ cells incubated with normal rabbit or the anti-plasminogen serum (diluted 1:50). An FITC-conjugated goat antibody to rabbit IgG was used as a second antibody. The horizontal bar (M1) denotes the area in which fluorescence intensity exceeded the maximal staining intensity of control unlabeled cells. Panels a and c: MIM and 25-12 cells, respectively, incubated with normal rabbit serum. Panels b and d: the same cells labeled with rabbit anti-plasminogen serum. A total of 5000 cells were analyzed for each sample.

FIG. 4 shows results of a Northern blot analysis for uPAR and uPA mRNA expression. 30 μg of total RNA were loaded onto each lane of a 1.1% formaldehyde-agarose gel (inset) and size-fractionated by electrophoresis. The blots were hybridized successively with a 0.77 kb genomic fragment of MMP-1, a uPAR cDNA, a 1.2 kb uPA cDNA fragment and a 0.8 kb fragment of rat cyclophilin cDNA. Laser densitometry was used to measure the intensity of the bands relative to control cyclophilin mRNA bands. Results of this analysis are shown in the bar graph. The values have been normalized relative to control, MIM cells which were assigned a value of 1.0. Shown in panel a are results for MMP-1 (solid bars) and uPAR (open bars). Shown in inset (left to right) are results obtained with MIM, clone 25-2 and clone 25-12 cells. Values for uPA are shown in panel b with RNA bands derived from (left to right) MIM and clone 25-12 cells.

FIG. 5 shows results of a flow cytometric analysis of uPAR expression. MIM and 25-12 cells (10⁵) were incubated with MAb 3936 to uPAR or with an isotype-matched monoclonal antibody to an irrelevant antigen (both at a concentration 5 μg/ml). An FITC-conjugated goat anti-mouse IgG was used as a second antibody. Panels a and b: MIM and 25-12 cells, respectively, incubated with control antibody (open histogram) and the same cells treated with anti-uPAR MAb 3936 (shaded histogram). Bar graphs shown in panel c represent the mean intensity of fluorescence (MIF) calculated for MIM and 25-12 cells and expressed relative to the respective controls.

FIG. 6 shows the nucleotide sequences of Exon 1 (SEQ. ID. NO.:1), Intron 1 (SEQ. ID. NO.:2) and Exon 2 (SEQ. ID. NO.:3) of an antisense having type I collagenase (MMP-1) inhibiting activity.

FIG. 7 illustrates the loss of tumorigenicity in melanoma cells expressing MMP-1 antisense mRNA.

### DETAILED DESCRIPTION OF THE INVENTION

Suppression of type I collagenase synthesis in human melanoma cells with antisense RNA significantly reduces proteolysis of type I and type IV collagen matrices in vitro, as described in Durko et al. (1997, Biochim. Biophys. Acta 1356, 271).

Because invasion and metastasis in vivo are dependent on the production of ECM-degrading enzymes, blocking of MMP-1 synthesis could provide a novel approach for inhibiting invasion in vivo.

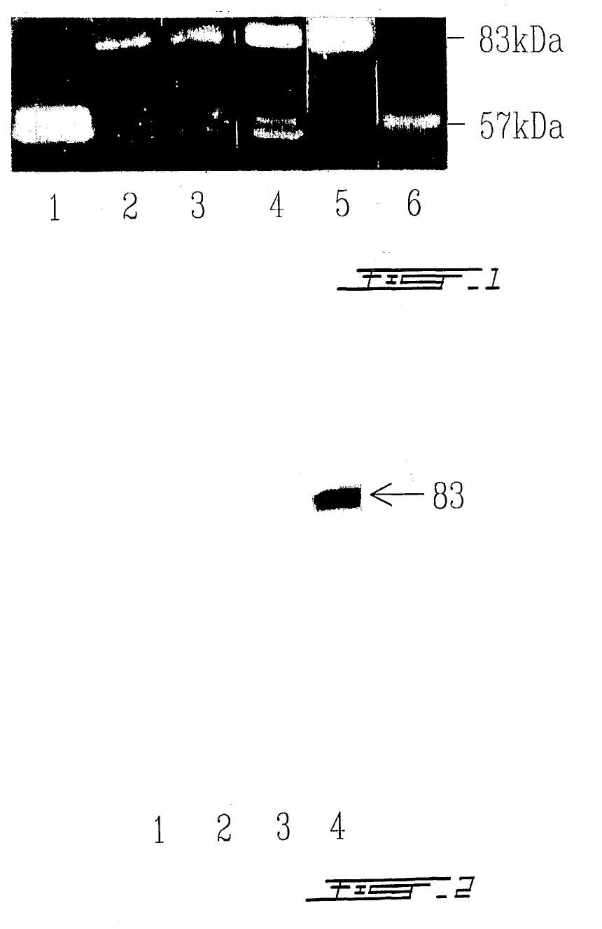

Plasmin is a major activator of the type I collagenase, the impact of type I collagenase suppression on the urokinase/plasmin system of proteolysis was therefore assessed. As shown in FIG. 1, gel zymography revealed the appearance of two new caseinolytic bands of Mr 81-83000 in conditioned media of type I collagenase-depleted, but not of wild-type cells and these were identified as plasmin bands. This increased extracellular plasmin activity coincided with reduced membrane-associated plasminogen levels and decreased expression of the urokinase-type plasminogen activator receptor at both the mRNA (up to 83% reduction) and cell-surface (up to 48% reduction) levels, while urokinase (uPA) mRNA levels remained unchanged. The results indicate that in these cells the urokinase/plasmin system is regulated by type I collagenase levels. It was therefore surprisingly found that melanoma cells in which MMP-1 production is suppressed have significantly reduced uPAR levels without uPA level alterations, decreased cell-surface bound plasminogen and correspondingly increased extracellular plasmin levels. The present findings that uPAR is downregulated when MMP-1 is suppressed, indicate that uPAR can be transcriptionally regulated by the levels of membrane-associated MMP-1. These findings add a new element to elucidate the complex relationship between these two systems of ECM-degrading proteinases. The proteolytic activities of MMP-1 and the plasminogen/plasmin system are therefore coordinated not only at the functional level but also at the level of transcriptional regulation. The existence of a regulatory link between MMP-1 levels and the expression and function of uPAR was a surprise discovery. The data presented herein identify MMP-1 as a putative regulator of uPAR/uPA-mediated proteolysis. This mechanism combined to the fact that uPAR synthesis is transcriptionally linked to the expression and function of the vitronectin receptor αᵥβ₃ (Nip, J., Rabbani, S. A., Shibata, H. R., and Brodt, P. (1995) J. Clin. Invest. 95, 2096-2103) provides a link between cellular adhesion, ECM turnover, motility and invasion. The transcriptional link discovered between MMP-1 and uPAR can therefore provide a mechanism for coordinating plasmin-mediated proteolysis and ECM turnover.

It was previously shown that the invasive human melanoma MIM cells constitutively express high levels of MMP-1 and MMP-2 and that suppression of MMP-1 expression in these cells markedly reduced their invasion through type IV collagen-containing matrices (Durko, M., Navab, R., Shibata, H., and Brodt, P. (1997) Biochim. Biophys. Acta, 1356, 271-280).

Referring to FIG. 1, to investigate plasmin production by the tumor cells, casein zymography was performed on conditioned media derived from MIM cells (lane 1), a sense-transfected clone 7-1 (Durko, M., Navab, R., Shibata, H., and Brodt, P. (1997) Biochim. Biophys. Acta, 1356, 271-280) (not shown) or clonal lines 25-2, 25-11 and 25-12 in which MMP-1 expression at the mRNA level was reduced by 90-96% (lanes 2-4).

While in all zymograms a doublet of 54-57 kDa corresponding to the molecular mass of stromelysin-1 was observed (Durko, M., Navab, R., Shibata, H., and Brodt, P. (1997) Biochim. Biophys. Acta, 1356, 271-280), only zymograms of the MMP-1-depleted clones 25-2, 25-11 and 25-12 but not of MIM or sense-transfected clone 7-1 (not shown) had two additional caseinolytic bands of 81-83 kDa. These caseinolytic activities could be blocked by the plasmin inhibitor Amino-n-Caproic acid (lane 6) but not by the metalloproteinase inhibitor EDTA (lane 5).

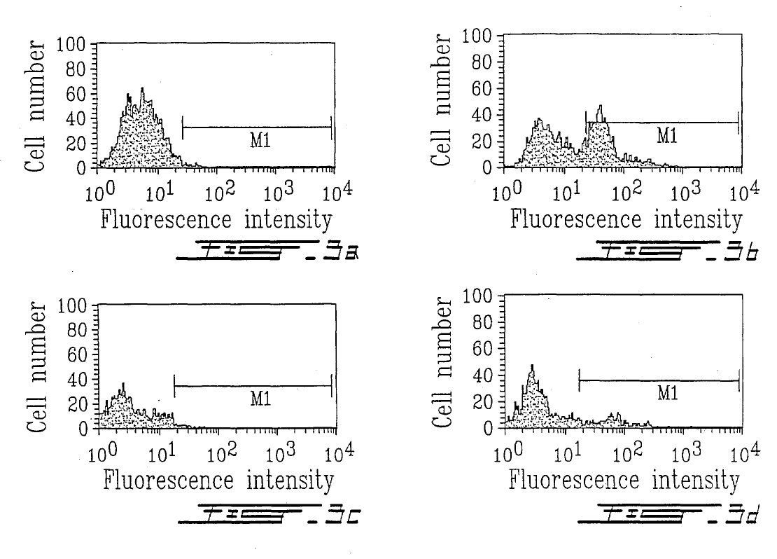

Referring to FIG. 2, Western blot analysis of plasmin production with a rabbit antiserum to plasminogen confirmed the presence in clone 25-11 conditioned medium (lane 3) of a 81-83 kDa doublet corresponding to the caseinolytic activities noted in the zymograms. The enzymatic activity associated with these bands suggest that they represent the activated forms of plasminogen, namely plasmin. Only a single Mᵣ 81000 band was detected in blots of wild type MIM conditioned medium (lanes 1 and 2) and it was considerably weaker (2.5×) than the corresponding band in blots of 25-11 conditioned medium (lane 3). Two bands of similar molecular masses corresponding to two forms of Lys₇₇ plasminogen (Castellino, F. J., and Powell, J. R. (1981) Methods Enzymol. 80, 365-378) were seen in blots of the purified enzyme which was used as a positive control (lane 4).

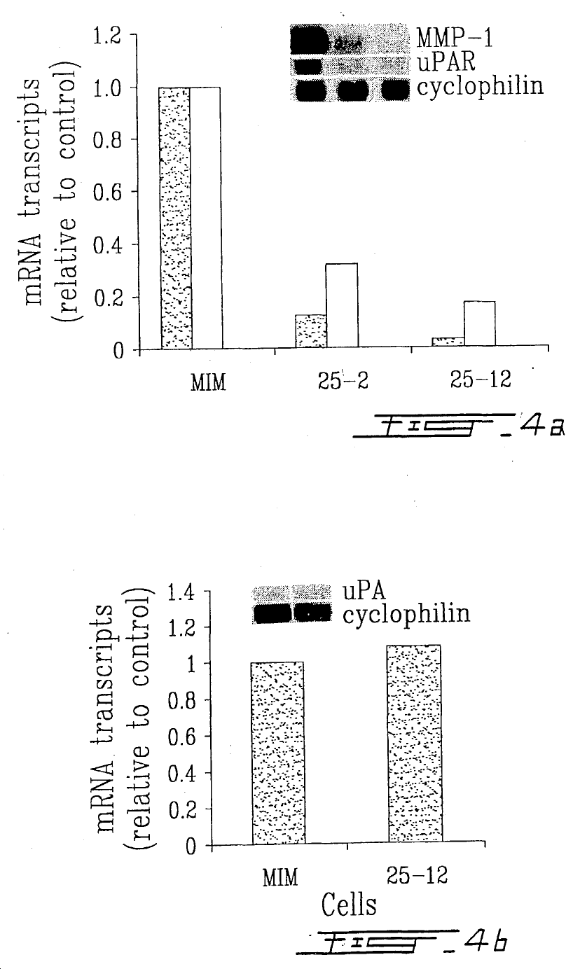

To determine whether the increased levels of plasmin in the conditioned medium coincided with a reduction in cell-bound plasmin, cell surface enzyme levels were measured by immuno-cytofluorometry. Results in FIG. 3 show that MMP-1-depleted 25-12 cells (panel d) had significantly reduced cell-surface associated plasmin levels as compared to the wild type MIM cells (panel b). This was reflected in a reduction of 67% in the number of positively-labeled cells.

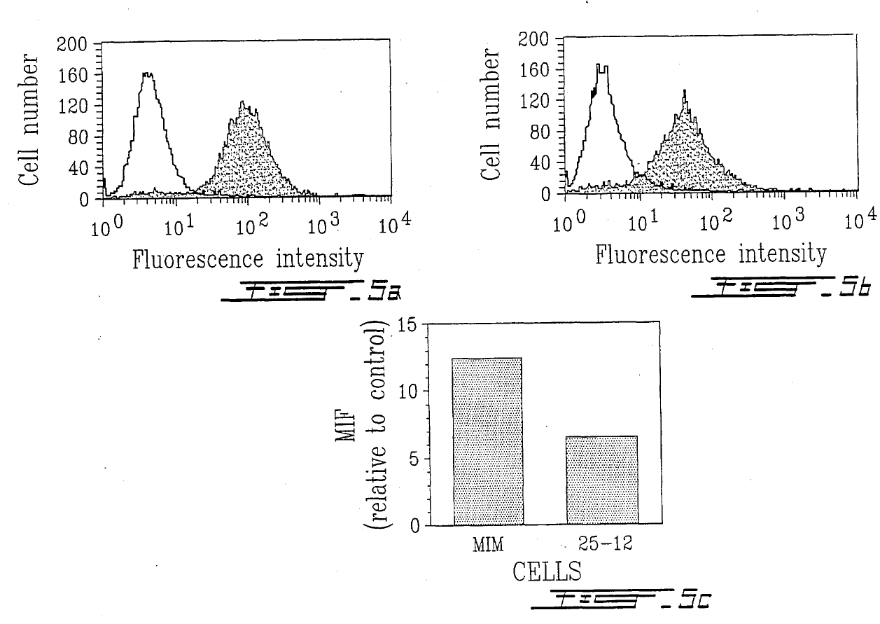

Following these findings, the expression in these cells of other components of the plasminogen/plasmin system, namely uPAR and uPA, were investigated. Using Northern blot analysis, as shown in FIG. 4, it was found that uPAR mRNA expression (panel a) in 25-2 and 25-12 cells decreased proportionally to the extent of MMP-1 suppression, resulting in a 3-5 fold reduction in uPAR mRNA transcripts in these cells relative to MIM cells. On the other hand, mRNA levels for uPA were not significantly altered (panel b).

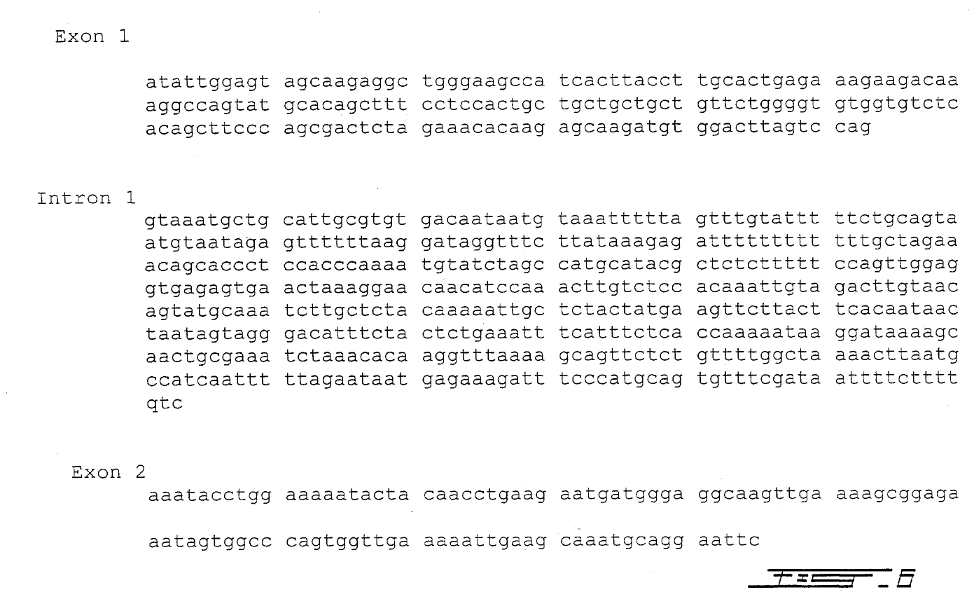

Referring to FIG. 5, the reduction in uPAR mRNA was reflected in a decrease in cell surface uPAR expression as demonstrated by immuno-cytofluorometry. When stained with MAb 3936 to uPAR, 25-12 cells showed a decrease of 48% in fluorescence intensity relative to wild-type MIM cells. Clone 25-2 cells in which uPAR mRNA levels were reduced by only 68% showed a reduction of 30% in fluorescence intensity compare to MIM cells (results not shown).

While plasmin activity in MIM conditioned medium was not detectable by casein zymography (FIG. 1, lane 1), a weak, 81 kDa band was observed when the same conditioned medium was analyzed by Western blotting (FIG. 2, lane 2). This apparent discrepancy may be due to differences in the sensitivity of the two assay systems as the levels of plasmin released by MIM cells may have been below the threshold necessary for detection of caseinolytic activity. The appearance of plasmin in the conditioned medium of MMP-1 -depleted cells suggests, in turn, that plasminogen/plasmin conversion can still occur on the surface of these cells despite the greatly reduced uPAR levels, but the enzyme may then be rapidly released. Alternatively, it is possible that plasminogen activation occurs in the conditioned medium after it is released from the cell surface.

The source of MIM-associated plasminogen has not been determined. Binding of exogenous plasminogen from bovine serum present in the culture medium has been reported for other human cells (Meissauer, A., Kramer, M. D., Schirrmacher, V., and Brunner, G. (1992) Exp. Cell Res. 199, 179-190). Extrahepatic production of plasminogen has also been reported (Saksela, O., and Vihko, K. K. (1986) FEBS Lett. 204, 193-197) and the possibility that MIM cells produce plasminogen endogenously can therefore not be ruled out. Using RT-PCR, preliminary evidence for plasminogen mRNA transcripts in clone 25-2 cells was obtained, suggesting that in these cells all the components of the plasminogen system may be autonomously produced.

The reduction in cell-bound plasmin and concomitant increase in extracellular plasmin levels seen in MMP-1 - depleted cells could have been the result of changes in uPAR expression and plasminogen/plasmin conversion. While this conversion is known to take place at the cell surface and there is evidence for plasminogen binding sites on the surface of normal and transformed cells (Meissauer, A., Kramer, M. D., Schirrmacher, V., and Brunner, G. (1992) Exp. Cell Res. 199, 179-190; Saksela, O., and Vihko, K. K. (1986) FEBS Lett. 204, 193-197), the plasminogen receptor has not yet been identified. Recent studies have suggested that annexin 11 (Hajjar, K. A., Jacovina, A. T., and Chacko, J. (1994) J. Biol. Chem. 269, 21191-21197) may be a co-receptor for plasminogen and tissue type plasminogen activator (t-PA) on endothelial cells but it is unclear whether it also plays this role in other cells. In cells expressing uPA such as leukemic cells and fibroblasts, uPA and plasminogen have been shown to associate with distinct binding sites. Interestingly however, in the latter, the binding of uPA to its receptor was shown to result in increased cell surface binding of plasminogen and this was attributed to uPAR-induced alterations in the plasminogen binding sites (Plow, E. F., Freaney, D. E., Plescia, J., and Miles, L. A. (1986) J. Cell. Biol. 103, 2411-2420). It is therefore conceivable that the uPAR-uPA complex has a stabilizing effect on the putative plasminogen receptor or on its association with plasminogen. Retention of plasmin on the cell surface may in turn be required for optimal catalytic activity because once released into the extracellular environment this activity can be blocked by circulating inhibitors (Meissauer, A., Kramer, M. D., Schirrmacher, V., and Brunner, G. (1992) Exp. Cell Res. 199, 179-190; Plow, E. F., Freaney, D. E., Plescia, J., and Miles, L. A. (1986) J. Cell. Biol. 103, 2411-2420). When uPAR levels are significantly reduced, as is the case in MMP-1 suppressed cells, this may destabilize the plasminogen/“receptor” complex resulting in increased plasmin release. Alternatively, MMP-1 levels may have a direct effect on the putative “plasminogen receptor” or MMP-1 may regulate plasmin retention through its role in extracellular matrix turnover.

MMP-1 and uPAR transcription are co-regulated by growth factors such as EGF, cytokines such as IL-1 and phorbol esters (Durko, M., and Brodt, P. (1996) In Cell Adhesion and Invasion in Cancer Metastasis. (Brodt, P. Ed.), pp. 113-150, R. G. Landes Company, Medical Intelligence Unit, Georgetown, Tex.). This coordinated expression may be essential for MMP-1 function because the metalloproteinase depends on cell-surface generated plasmin for its activation. The present findings that uPAR is downregulated when MMP-1 is suppressed suggest that uPAR transcription may also be regulated by the levels of plasma membrane-associated MMP-1. The regulatory mechanism linking these two molecules can involve a putative cell-surface MMP-1 receptor (Moll, U. M., Lane, B., Zucker, S., Suzuki, K., and Nagase, H. (1990) Cancer Res. 50, 6995-7002; Brooks, P. C., Stromblad, S., Sanders, L. C., von Schalscha, T. L., Aimes, R. T., Stetler-Stevenson, W. G., Quigley, J. P., and Cheresh, D. A. (1996) Cell 85, 683-693) akin to the integrin vitronectin receptor αᵥβ₃ which was identified as a receptor for MMP-2 (Brooks, P. C., Stromblad, S., Sanders, L. C., von Schalscha, T. L., Aimes, R. T., Stetler-Stevenson, W. G., Quigley, J. P., and Cheresh, D. A. (1996) Cell 85, 683-693). uPAR has been described in association with integrins, and integrin-dependent signaling was shown to regulate the expression of components of the urokinase-uPAR system (Nip, J., Rabbani, S. A., Shibata, H. R., and Brodt, P. (1995) J. Clin. Invest. 95, 2096-2103; Chapman, H. A. (1997) Curr. Opin. Cell Biol. 9, 714-724). Alternatively, products of MMP-1-mediated ECM degradation such as ECM protein fragments or ECM bound growth factors could regulate uPAR expression. It should be noted that uPAR and the uPAR/uPA complex have been identified as receptors for the ECM protein vitronectin (Stahl, A., and Mueller, B. M. (1997) Int. J. Cancer 71, 116-122), while plasmin can mediate cell detachment from the ECM.

The present invention will be more readily understood by referring to the following examples which are given to illustrate the invention rather than to limit its scope.

The following probes were used: the 0.77 kb MMP-1 DNA which was originally cloned in the pSVk3 plasmid as previously described (Durko, M., Navab, R., Shibata, H., and Brodt, P. (1997) Biochim. Biophys. Acta, 1356, 271-280; Goldberg, G. I., Wilhelm, S. M., Kronberger, A., Bauer, E. A., Grant, G. A., and Eisen, A. Z. (1986) J. Biol. Chem. 261, 6600-6605), the full-length uPAR cDNA (Roldan, A. L., Cubellis, M. V., Masucci, M. T., Behrendt, N., Lund, L. R., Dano, K., Appella, E., and Blasi, F. (1990) EMBO J. 9, 467-474), a 1.2 kb uPA cDNA fragment and a 0.8 kb fragment of rat cyclophilin cDNA (Danielson, P. E., Forss-Petter, S., Brow, M. A., Calavetta, L., Douglass, J., Milner, R. J., and Sutcliffe, J. G. (1988) DNA 7, 261-267) all from Dr. S. A. Rabbani (Royal Victoria Hospital, Montreal, Canada).

The antibodies used included a rabbit antiserum recognizing plasminogen and plasmin from Dr. L. A. Moroz (Royal Victoria Hospital, Montreal, Canada) and the mouse monoclonal antibody 3936 which recognizes the human uPAR in its ligand-bound or unbound forms (American Diagnostica Inc., Greenwich, Conn.). Secondary antibodies were an alkaline phosphatase-conjugated goat anti-rabbit IgG (Jackson ImmunoResearch Laboratories, Inc., West Grove, Pa.), an FITC-conjugated goat anti-rabbit IgG (Jackson ImmunoResearch Laboratories, Inc., West Grove, Pa.), and an FITC-conjugated goat anti-mouse IgG (Jackson ImmunoResearch Laboratories, Inc., West Grove, Pa.).

The MIM cell line was established from an inguinal lymph node metastasis of a male melanoma patient as previously described (Nip, J., Shibata, H., Loskutoff, D. J., Cheresh, D. A., and Brodt, P. (1992) J. Clin. Invest. 90, 1406-1413). Cells were maintained as a monolayer culture in RPMI 1640 medium supplemented with 5% heat-inactivated fetal bovine serum (FCS), 2 mM glutamine, penicillin (100 units/ml) and streptomycin (100 μg/ml). At confluence, the cells were dispersed with a 0.5 mM EDTA solution and replated at a dilution of 1:5. Clonal lines were derived from MIM cells stably transfected with the pSVk3 plasmid vector (Pharmacia) expressing a 777 bp genomic DNA fragment of MMP-1 (Durko, M., Navab, R., Shibata, H., and Brodt, P. (1997) Biochim. Biophys. Acta, 1356, 271-280; Collier, I. E., Smith, J., Kronberger, A., Bauer, E. A., Wilhelm, S. M., Eisen, A. Z., and Goldberg, G. I. (1988) J. Biol. Chem. 263, 10711-10713) in the sense (clone 7-1) or antisense orientation (clones 25-2, 25-11 and 25-12) relative to the SV40 origin and early promoter region, as described in detail previously (Durko, M., Navab, R., Shibata, H., and Brodt, P. (1997) Biochim. Biophys. Acta, 1356, 271-280).

### EXAMPLE I

### RNA Extraction and Northern Blot Analysis

RNA isolation and Northern blot analysis were carried out using standard protocols (Durko, M., Navab, R., Shibata, H., and Brodt, P. (1997) Biochim. Biophys. Acta, 1356, 271-280). cDNA probes were labeled by the random primer extension labeling method using [α-³²P] dCTP (DuPont). Prehybridization and hybridization of the nylon Hybond™-N membranes (Amersham Life Science) were at 42° C. and washings at 55° C. The blots were radioautographed at −80° C. The relative amounts of mRNA transcripts were analyzed by laser densitometry using an Ultroscan XL Enhanced Laser Densitometer (LKB Instruments Inc., Bromma, Sweden) and normalized relative to cyclophilin controls.

### EXAMPLE II

### Gel Zymography

Cell cultures were grown to 80% confluency, washed twice with serum-free RPMI and cultured in serum-free RPMI medium for 48-72 hr. The supernatants were collected, filtered to remove debris and then dialyzed against a “collagenase” buffer (1 mM Tris-HCl, pH 7.6 with 1 mM CaCl₂) for 24 hr, aliquoted, concentrated by freeze-drying at 31 120° C. using a cooling trap (HETOTRAP CT 110) and stored at −20° C. until used. The concentrated conditioned media were mixed with the SDS sample buffer and the proteins separated by electrophoresis on 10% SDS-polyacrylamide gels which were copolymerized with 1 mg/ml of casein. The gels were then washed for 1 hr in a solution of 2.5% Triton X100. For the enzymatic reaction to occur, the gels were incubated for 18 hr at 37° C. (with shaking) in a solution of 50 mM Tris-HCl, pH 8 containing 10 mM CaCl₂. For some of the gels, 20 mM EDTA (a metalloproteinase inhibitor) or 500 μg/ml Amino-n-Caproic acid (a plasmin inhibitor) were added to the buffer. The gels were stained in 0.5% Coomassie blue R250 and destained in 20% methanol and 10% acetic acid until the clear bands of lysis appeared.

### EXAMPLE IlIl

### Western Blot Analysis

Concentrated (50×), serum-free conditioned media and purified plasminogen (from Dr. L. A. Moroz, Royal Victoria Hospital, Montreal, Quebec) were separated by electrophoresis on a 10% SDS-polyacrylamide gel under nonreducing conditions and the proteins electrophoretically transferred onto nitrocellulose filters (0.2 mm; Schleicher and Schuell). The blots were probed with a rabbit antiserum to plasminogen/plasmin at a dilution 1:50. Alkaline phosphatase-conjugated goat anti-rabbit IgG at a dilution of 1:2000 was used as a second antibody.

### EXAMPLE IV

### Immuno-Cytofluorometry

Cells were cultured in serum-free medium for 48 hr, dispersed and 10⁵ cells then incubated for 1 hr on ice with 100 μl of rabbit antiserum to plasminogen/plasmin or a mouse monoclonal antibody to uPAR both diluted in PBS containing 0.1% BSA. Non-immune sera or an irrelevant isotype-matched mouse monoclonal antibody were used as controls. After extensive washing with cold buffer the cells were incubated with FITC-conjugated goat anti-rabbit or anti-mouse IgG (diluted 1:100) for 1 hr on ice, washed and fixed in PBS containing 1% formalin. The labeled cells were analyzed by flow cytofluorometry using a FACSCalibur™ System (Becton-Dickinson, San Jose, Calif.).

### EXAMPLE V

### In Vivo Data

Fourteen week old female nude mice were injected intradermally with 5×10⁵ human melanoma MIM cells or clone MIM/25-12 cells expressing MMP-1 antisense mRNA. Tumor growth was monitored twice weekly and the tumors were measured using a caliper. Measurements were in two planes and the average of the two measurements recorded. Results are expressed as the mean tumor size based on 3 mice per group (FIG. 7). All animals injected with MIM cells developed tumors by day 25 post injection and all were moribund day 67 with large tumors and lymph node metastases. Only one animal injected with MIM/25-12 cells developed a tumor (day 79). Animals were observed for 14 weeks at which time 2/3 animals injected with MIM/25-12 cells were still alive and tumor free.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An antisense having type I collagenase (MMP-1) synthesis-inhibiting activity or a functional analog thereof, which comprises a first nucleotide sequence adapted to hybridize with a cellular RNA transcribed from a second nucleotide sequence encoding a peptide having MMP-1 activity, for substantially reducing MMP-1 synthesis or MMP-1 function and/or inhibiting invasion of an extracellular matrix by a tumor cell in a patient.

2. An antisense according to claim 1, wherein said first nucleotide sequence has from about 18 nucleotides to about 770 nucleotides and is complementary to a target region of said RNA encoding said peptide having MMP-1 activity

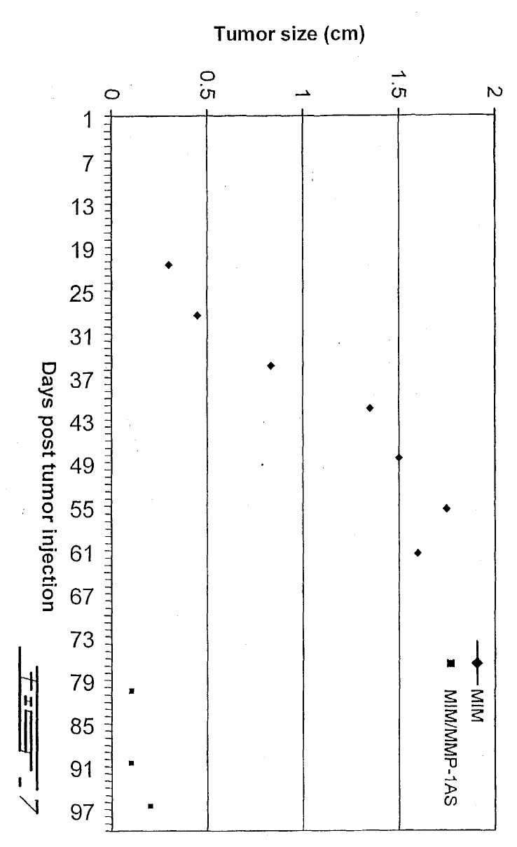

3. An antisense according to claim 2, wherein said first nucleotide sequence is as set forth in FIG. 6 (SEQ. ID. NO:1).

4. An expression vector comprising an antisense according to claim 1 operably linked to a promoter region.

5. A method for substantially inhibiting tumor cell invasion of an extracellular matrix in a patient, comprising substantially inhibiting expression or function of type I collagenase (MMP-1) by said tumor cell, thereby substantially inhibiting degradation of types I, II, III and IV collagen of said extracellular matrix by said patient tumor cell.

6. A method for substantially inhibiting tumor cell invasion of an extracellular matrix in a patient, comprising substantially inhibiting expression or function of type I collagenase (MMP-1) by said tumor cell, thereby substantially inhibiting degradation of types I, II, III and IV collagen of said extracellular matrix by said patient tumor cell, wherein said method further comprises administering to said patient an antisense according to claim 1.

7. A method according to claim 6, wherein said tumor cell is a melanoma cell or other tumor types where MMP-1 and uPAR were implicated in invasion.

8. The method of claim 7, wherein said other tumor types are selected from the group consisting of breast carcinoma and osteosarcoma.

9. An antisense having type I collagenase (MMP-1) inhibiting activity or a functional analog thereof, which comprises a nucleotide sequence designed to hybridize with a cellular RNA transcribed from a genomic DNA sequence coding for a peptide having MMP-1 activity, for substantially inhibiting plasmin-mediated proteolysis of an extracellular matrix by a tumor cell or normal, tumor-associated stromal and endothelial cells in a patient.

10. An antisense according to claim 9, wherein said first nucleotide sequence has from about 18 nucleotides to about 770 nucleotides and is complementary to a target region of said RNA encoding a peptide having MMP-1 activity.

11. An antisense according to claim 10, wherein said first nucleotide sequence is as set forth in SEQ ID NO:1.

12. A pharmaceutical composition comprising an antisense according to claim 9, in association with a pharmaceutically acceptable carrier.

13. A method for substantially inhibiting plasmin-mediated proteolysis of an extracellular matrix in a patient by a tumor cell, comprising suppressing expression of type I collagenase (MMP-1) by said patient tumor cell, thereby substantially inhibiting tumor cell invasion of said extracellular matrix in said patient.

14. A method for substantially inhibiting plasmin-mediated proteolysis of an extracellular matrix in a patient by a tumor cell, comprising suppressing expression of type I collagenase (MMP-1) by said patient tumor cell, thereby substantially inhibiting tumor cell invasion of said extracellular matrix in said patient, wherein said method further comprises, comprising administering to said patient an antisense according to claim 9.

15. A method according to claim 14, wherein said tumor cell is a melanoma cell.

16. A method for substantially inhibiting MMP-1 and plasmin-mediated migration and invasion by vascular endothelial cells comprising suppressing expression of type 1 collagenase (MMP-1) in a tumor cell thereby blocking angiogenesis.
